(19) United States
(12) Patent Application Publication
(10) Pub. No.: US 2008/0131914 A1
(43) Pub. Date: Jun. 5, 2008

(54) ASSESSING RISK OF CEREBROVASCULAR THROMBOSIS BY MEASURING C4D

(76) Inventors: Joseph M. Ahearn, Sewickley, PA (US); Susan M. Manzi, Wexford, PA (US); Amy Kao, Pittsburgh, PA (US)

Correspondence Address: Gwen R. Wood, Ph.D., J.D.;Eckert Seamans Cherin & Mellott, LLC, 44th Floor, 600 Grant Street, Pittsburgh, PA, 15219, US

(21) Appl. No.: 11/939,188

(22) Filed: Nov. 13, 2007

Related U.S. Application Data
(63) Continuation-in-part of application No. 11/521,643, filed on Sep. 14, 2006.
(60) Provisional application No. 60/717,525, filed on Sep. 14, 2005.

(51) Int. Cl. G01N 33/567; G06F 19/00

(52) U.S. Cl. 435/7.21; 702/21

(57) ABSTRACT

The present invention provides methods for the convenient, economical and sensitive assessment of the risk of whether a person will suffer from a thrombotic stroke. The present invention also provides methods for diagnosing or monitoring cerebrovascular thrombosis in a person as well as assessing the degree of severity of a cerebrovascular thrombosis in a person. More specifically, it has been determined that persons at risk of, or has been afflicted with, a cerebrovascular thrombosis have an elevated deposition of C4d on their platelets.

This application is a continuation-in-part application of U.S. application Ser. No. 11/521,643, filed Sep. 14, 2006, which claims priority to U.S. Provisional Application No. 60/717,525, filed Sep. 14, 2005, the contents of which are incorporated herein by reference in their entirety.

### BACKGROUND OF THE INVENTION

1. Field of the Invention

The present invention relates to the field of cerebrovascular thromboses and, more particularly, to the identifying of biomarkers for assessing the risk, for diagnosing and for determining the severity of cerebrovascular thromboses in individuals.

2. Description of the Prior Art

After heart disease and cancers, stroke is the third leading cause of death in the developed countries. Each year in the United States, about 700,000 new or recurring strokes take place. Every 45 seconds, someone in this country suffers from a stroke. On average, we have a one-in-five chance to suffer from a stroke during our lifetime.

Besides the high prevalence, the result of a stroke can be devastating: about 30% of all strokes are fatal; 50-70% of survivors will have a mild disability; 15-30% of survivors will become severely disabled; and up to 20% of survivors will require institutional care for at least three month post-stroke. The cost of strokes is not just measured in the billions of dollars lost in work, hospitalization, and the care of survivors in nursing homes. The major cost or impact of a stroke is the loss of independence that occurs in 30% of the survivors. What was a self-sustaining and enjoyable lifestyle may lose most, if not all, of its quality after a stroke and other family members can find themselves in a new role as caregivers.

Although modern medicine provides advanced approaches for stroke diagnosis and treatment, there remains a need for new methods to rapidly, conveniently, and effectively identify individuals with high risk of stroke and to diagnose or monitor the condition in patients already with some symptoms of a stroke. The present invention meets this and other related needs.

### SUMMARY OF THE INVENTION

In one aspect, this invention provides a method for assessing risk of cerebrovascular thrombosis in an individual. This method comprises the following steps: first, determining the platelet surface level of a complement pathway component C4d in the individual, and second, comparing the level of C4d of the individual with a standard control. In the event an increase in the level of C4d from the standard control is detected, an increased risk of cerebrovascular thrombosis is then indicated in this individual.

In some embodiments, the level of C4d is determined using an antibody that specifically binds C4d. Preferably, the C4d antibody is labeled, e.g., with a fluorescent moiety, which allows detection of the antibody by flow cytometry.

In other embodiments, the individual being tested has no clinical symptoms of cerebrovascular thrombosis; or the individual might have previously suffered from cerebrovascular thrombosis.

In another aspect, this invention provides a method for diagnosing or monitoring cerebrovascular thrombosis in an individual. This method comprises the following steps: first, determining the platelet surface level of a complement pathway component C4d in the individual, and second, comparing the level of C4d of the individual with a standard control. In the case an increase in the level of C4d from the standard control is detected, it is indicated that the individual is suffering from the condition of cerebrovascular thrombosis or a worsening of the condition.

In some embodiments, the level of C4d is determined using an antibody that specifically binds C4d. Preferably, the C4d antibody is labeled, e.g., with a fluorescent moiety, which allows detection of the antibody by flow cytometry.

In another embodiment, the individual being tested already has one or more clinical symptoms of cerebrovascular thrombosis.

In a further aspect, the present invention provides a method of assessing the severity of cerebrovascular thrombosis in an individual. This method comprises the following steps: first, determining the platelet surface level of a complement pathway component 4 C4d in the individual and, second, comparing the level of C4d of the individual with a standard control, wherein the magnitude of increase in the level of C4d from the standard control correlates with the severity of cerebrovascular thrombosis in the individual.

In yet another aspect, this invention provides a computer readable medium for predicting, diagnosing, or monitoring cerebrovascular thrombosis in an individual. This computer readable medium comprises: (a) code for receiving data corresponding to a level of the complement pathway component C4d on the surface of platelets from an individual; (b) code for retrieving a standard control; and (c) code for comparing the data in (a) with the standard control in (b).

### DEFINITIONS

As used herein, the term “cerebrovascular thrombosis” refers to a pathological process in which a blood clot builds up in a blood vessel in the brain and ultimately causes blockage of the blood vessel, which in turn leads to an ischemic stroke, a type of stroke characterized by damage to the brain cells due to insufficient blood flow and oxygen supply. In this application, this term encompasses any type of ischemic stroke, including the so-called transient ischemic attack (TIA). The clinical symptoms of cerebrovascular thrombosis include, but are not limited to, weakness or paralysis on one side of the body; a partial or complete loss of voluntary movement and/or sensation in a leg and/or arm; speech problems and weak muscles of the face, which can cause drooling; numbness or tingling in the leg, arm, or face; impaired balance, vision, and swallowing functions; difficulty breathing and even unconsciousness.

As used herein the “complement pathway” or “complement system” refers to a complex network of more than 30 functionally linked proteins that interact in a highly regulated manner to provide many of the effector functions of humoral immunity and inflammation, thereby serving as the major defense mechanism against bacterial and fungal infections. This system of proteins acts against invasion by foreign organisms via three distinct pathways: the classical pathway (in the presence of antibody), the alternative pathway (in the absence of antibody), and the lectin pathway. Once activated, the proteins within each pathway form a cascade involving sequential self-assembly into multimolecular complexes that perform various functions intended to eradicate the foreign antigens that initiated the response. For a review of the complement pathway, see, e.g., Sim and Tsiftsoglou, Biochem. Soc. Trans. 32:21-27 (2004).

The classical pathway is usually triggered by an antibody bound to a foreign particle. It consists of several components that are specific to the classical pathway and designated C1, C4, C2. Sequentially, binding of Clq to an antigen-antibody complex results in activation of C 1 r and Cis (both are serine proteases), and activated C1s cleaves C4 and C2 into, respectively, C4a and C4b and C2a and C2b. Fragments C4b and C2a assemble to form C4b2a, which cleaves protein C3 into C3a and C3b, which completes activation of the classical pathway. Fragments C4b and C3b are subject to further degradation by Factor I. This factor cleaves C4b to generate C4d and also cleaves C3b, to generate iC3b followed by C3d. Thus, activation of the classical pathway of complement can lead to deposition of a number of fragments, such as C4d, iC3b, and C3d, on immune complexes or other target surfaces. Such targets include cells circulating in the blood, e.g., lymphocytes and other white blood cells, erythrocytes, and platelets.

Components of the complement pathway include proteins C1, C4, C2, C3, and fragments thereof, e.g., C1 q, C1 r, CI s, C4a, C4b, C2a, C2b, C4b2a, C3a, C3b, C4c, C4d, iC3b, C3d, C3i, C3dg. Also included are C5, C5b, C6, C7, C8, C9, Clinh, MASP2, CR1, DAF, MCP, CD59, C3aR, C5aR, ClqR, CR2, CR3, and CR4, as well as other complement pathway components, receptors and ligands not listed specifically herein.

As used herein, “the platelet surface level of a complement pathway component C4d” refers to the amount of C4d found on the surface of a predetermined number of platelets obtained from an individual person.

As used herein, a “standard control” refers to a platelet surface C4d level used as a comparison basis in practicing a method of the present invention. Such a standard control should reasonably indicate the level of platelet surface C4d in an average individual who is not suffering from or at risk of developing cerebrovascular thrombosis, and is not suffering from or at risk of any other diseases or conditions that tend to elevate platelet surface C4d level. Preferably, a standard control reflects the platelet surface C4d level from a healthy individual with medical background, as well as in age, gender, ethnicity, etc., comparable to the individual whose platelet surface C4d level is being tested.

As used herein “an increase in the level of C4d from the standard control” refers to a positive change in value from the standard control.

As used herein, an “antibody” refers to a glycoprotein of the immunoglobulin family or a polypeptide comprising fragments of an immunoglobulin that is capable of noncovalently, reversibly, and in a specific manner binding a corresponding antigen. The typical antibody structural unit is a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, and μ constant region genes, as well as the myriad immunoglobulin variable region genes. Light chains are classified as either κ or λ. Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (VL) and variable heavy chain (VH) refer to these regions of light and heavy chains respectively.

The term antibody, as used herein, includes both monoclonal and polyclonal antibodies, and encompasses antibodies raised in vivo, e.g., produced by an animal upon immunization by an antigen, and antibodies generated in vitro, e.g., generated by hybridomas. As used in this application, antibodies that specifically recognize the same antigen, e.g., a pathogenic organism, are regarded as “one antibody,” regardless of whether they actually bind to the same or to separate antigenic epitopes of the antigen.

For preparation of monoclonal or polyclonal antibodies, any technique known in the art can be used (see, e.g., Kohler & Milstein, Nature 256:495-497, 1975; Kozbor et al., Immunology Today 4:72, 1983; Cole et al., Monoclonal Antibodies and Cancer Therapy, pp. 77-96. Alan R. Liss, Inc., 1985). Techniques for the production of single chain antibodies (see, e.g., U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., supra; Marks et al., Biotechnology, 10:779-783, 1992).

As used herein, the term “specific binding” or “specifically binds,” when used to describe the binding reaction between an antibody to a protein (e.g., C4d), refers to the characteristic of the binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to a component of the complement pathway or to a surface marker of platelets, polymorphic variants, alleles, orthologs, and conservatively modified variants, or splice variants, or portions thereof, can be selected to obtain only those polyclonal antibodies that are specifically immunoreactive with the component of the complement pathway or the platelet surface marker and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

### DESCRIPTION OF THE PREFERRED EMBODIMENTS

### I. Introduction

The present invention provides a novel method for expediently assessing a person's risk for a future episode of cerebrovascular thrombosis, a type of stroke caused by the blockage of a blood vessel in the brain. According to this method, a patient's blood sample is taken and the level of platelet surface C4d, a component of the complement system, is measured. This C4d level is then compared with an established standard control, which reflects the average amount of platelet surface C4d found in a healthy person not at risk of cerebrovascular thrombosis or other conditions that tend to elevate C4d level on the platelets.

An increase in the C4d level indicates a heightened risk for an individual to suffer from cerebrovascular thrombosis. Similarly, the platelet surface C4d levels can also be used to diagnose cerebrovascular thrombosis or monitor the condition of cerebrovascular thrombosis in a patient, where a higher-than-normal C4d level indicates that presence of cerebrovascular thrombosis or a worsening of the condition in a patient.

### II. Determination of Platelet Surface C4d Level

The present invention involves conducting assays on platelets obtained from an individual to determine the level of C4d, a complement pathway component, deposited on the surface of platelets. The C4d level then is used to predict, diagnose, or monitor cerebrovascular thrombosis in the individual.

The procedure of determining platelet surface C4d level begins with acquisition of a blood sample from a patient. Generally, patient blood samples are treated with EDTA (ethylenediaminetetraacetate) to inhibit complement activation, and can be maintained at room temperature or under cold conditions. Assays are run preferably within 24 hours from sample collection.

Many methods for isolating platelets are known in the art. For instance, density gradient centrifugation methods are frequently used, where platelets are separated from other components of the blood based on difference in density following one or more rounds of centrifugation process. Isolation of platelets can also be achieved by an affinity-based method. For example, an antibody specific to a platelet surface marker (e.g., CD42b) can be first attached to a solid support (such as a Sepharose column), then contacted with a blood sample under conditions that permits the formation of antigen-antibody complex. While other undesired blood components are washed away, platelets can be recovered from the solid support.

In some embodiments of the present invention, fluorescence activated cell sorting, or FACS, is used to determine the number of platelets while other blood components are present in a sample. FACS is a technique used to separate cells according to their surface content of particular molecules of interest. A molecule of interest can be specific for a type of cell or for particular cell state. The molecule of interest can be fluorescently labeled directly by binding to a fluorescent dye, or by binding to a second molecule, which has been fluorescently labeled, e.g., an antibody, lectin, or aptamer that has been fluorescently labeled and that specifically binds to the molecule of interest. Thus, platelet specific markers can by used to distinguish platelets from other components of the blood such as red or white blood cells in a blood sample.

Isolation of platelets also refers to gating techniques used to assay platelets during flow cytometric analysis. A labeled marker specific for platelets (e.g., a labeled anti-CD42b monoclonal antibody) is used to determine the amount of platelets in a sample. A second labeled marker (e.g., a labeled anti-C4d antibody) is then used to determine the level of C4d on the surface of the platelets.

In one embodiment of the present invention, platelet surface C4d level is determined to predict, diagnose, or monitor the progression of cerebrovascular thrombosis in individuals.

The platelets are isolated or detected using platelet-specific antibodies e.g., anti-CD42b antibodies. In some embodiments, determination of the level of platelet surface C4d may be achieved by a number of methods including flow cytometry, ELISA using purified platelet preparations, and radioimmunoassay. In one embodiment of this invention, the determination of the platelet surface C4d level is made using flow cytometric methods, with measurements taken by direct or indirect immunofluorescence using polyclonal or monoclonal antibodies specific for C4d. The mean fluorescence channel (MFC) for the platelet surface C4d can be determined. Detection and quantification of C4d on the surface of platelets is described in, e.g., WO 04/093647 and U.S. Ser. No. 60/463,447, both of which are herein incorporated in their entirety by reference for all purposes.

### III. Kits

Kits for conducting the assays for predicting, diagnosing, or monitoring of cerebrovascular thrombosis in a patient are a part of this invention. The kits may comprise any of the various reagents needed to perform the methods described herein. For example, a kit adapted for the immunofluorescence assays generally comprises a conjugate of a monoclonal antibody specific for C4d with a first fluorescent moiety, and preferably also a conjugate of a monoclonal antibody specific for a platelet surface marker (e.g., CD42b) with a second, different fluorescent moiety. Additionally, the kits can comprise instructional material for the user and such other material as may be needed in carrying out assays of this type, for example, buffers, radiolabelled antibodies, colorimeter reagents, etc.

The antibodies for use in these methods and kits are known in the art and available commercially. For example, monoclonal antibodies specific for CD42b are available from commercial suppliers such as BIODESIGN International (Saco, Me.) and Yorkshire Bioscience (United Kingdom). Anti-C4d antibodies are available from Quidel Corp. (San Diego, Calif.) and are generally described in Rogers, J., N. Cooper, et al. PNAS 89:10016-10020 (1992); Schwab, C. et al. Brain Res. 707(2):196 (1996); Gemmell, C. J. Biomed. Mater. Res. 37:474-480 (1997); and, Stoltzner, S. E., et al. Am. J. Path. 156:489-499 (2000).

### IV. Diagnosis and Monitoring

### A. Diagnosis

Diagnosis of a patient with cerebrovascular thrombosis or with an increased risk of developing cerebrovascular thrombosis is carried out by comparing the level of platelet surface C4d in this patient with a base value or standard control for the quantity of C4d that is typically present on the surface platelets in normal individuals. In normal individuals, the level of C4d on the surface of platelets is very low to not detectable. When using flow cytometric measurement with indirect immunofluorescence, the MFC of C4d on platelet surface of healthy individuals ranged from −1.17 to 0.87 (mean −0.39). In contrast, the MFC of platelet surface C4d in eight patients suffering from cerebrovascular thrombosis was observed to range from 2.18 to 16.5

### B. Monitoring of Patients

A particular feature of the methods of this invention is to indicate or reflect the progress of cerebrovascular thrombosis that has occurred in a patient during the preceding several weeks or even several months. It is possible, using the claimed methods, to identify the worsening of cerebrovascular thrombosis that has previously occurred, or to predict a subsequent occurrence of cerebrovascular thrombosis based on the persistence of elevated level of C4d deposited on the surface of platelets.

### V. Automation and Computer Software

The determination of platelet surface C4d level, and the diagnostic and monitoring methods described above can be carried out manually, but often are conveniently carried out using an automated system and/or equipment, in which the blood sample is analyzed automatically to make the necessary determination or determinations, and the comparison with a standard control or reference value is performed automatically, using computer software appropriate to that purpose.

Thus, in one aspect, the invention comprises a method for predicting, diagnosing, or monitoring cerebrovascular thrombosis in an individual comprising (a) automatically determining, in a blood sample from the individual containing platelets, the level of C4d deposited on surfaces of platelets in the sample, and (b) automatically comparing the platelet surface C4d level with a standard control or reference value that reflects the average C4d level found on a normal, healthy individual's platelets.

In another aspect, the invention comprises a method for predicting, diagnosing, or monitoring cerebrovascular thrombosis in an individual comprising (a) automatically determining, in a blood sample from the individual containing platelets, the level of C4d deposited on surface of platelets in the sample, and (b) automatically comparing C4d level with a standard control indicating the average C4d level found on a normal, healthy individual's platelets.

Computer software, or computer-readable media for use in the methods of this invention includes a computer readable medium, which comprises:

(1) code for receiving data corresponding to a level of C4d deposited on the surface platelets in a blood sample;

(2) code for retrieving a standard control, which indicate the average C4d level found on a normal, healthy individual's platelets; and

(3) code for comparing the data in (a) with the standard control of (b).

In some embodiments of the invention, more than one standard control may be stored in a memory associated with a digital computer. After data corresponding to the level of platelet surface C4d are obtained (e.g., from an appropriate analytical instrument), the digital computer may compare the C4d level with one or more appropriate standard controls. After this comparison takes place, the digital computer can automatically determine if the data corresponding to the C4d level are associated with cerebrovascular thrombosis.

In some embodiments of the invention, more than one C4d level may be stored in a memory associated with a digital computer. For instance, the platelet surface C4d level from a particular individual may be measured at different points in time for the purpose of monitoring the progress of cerebrovascular thrombosis. After new data corresponding to C4d levels are obtained (e.g., from an appropriate analytical instrument), the digital computer can compare the C4d levels with the appropriate standard control(s), and/or with the platelet C4d levels recorded at previous time points. After this comparison takes place, the digital computer can automatically determine if the data corresponding to the C4d levels indicate an improvement or deterioration of cerebrovascular thrombosis in the patient.

Accordingly, one aspect of the invention may be embodied by computer code that is executed by a digital computer. The digital computer may be a micro, mini, or large frame computer using any standard or specialized operating system such as a Windows™ based operating system. The code may be stored on any suitable computer readable media. Examples of computer readable media include magnetic, electronic, or optical disks, tapes, sticks, chips, etc. The code may also be written by those of ordinary skill in the art and in any suitable computer programming language including, C, C++, etc.

### EXAMPLES

The following examples are provided by way of illustration only and not by way of limitation. Those of skill in the art will readily recognize a variety of non-critical parameters that could be changed or modified to yield essentially the same or similar results.

### Example 1

### Measuring Platelet Surface C4d Level in Healthy Individuals—Establishing Standard Controls

Twenty-five healthy individuals were studied. As shown in Table I, C4d was not detected on the surface of platelets in each of the twenty-five healthy individuals. Samples of 1 mL of EDTA-anticoagulated peripheral blood were taken from each individual and used as a source of platelets. The platelets were washed and resuspended in FACS buffer. Levels of C4d and CD42b were measured by two color indirect immunofluorescence using monoclonal antibodies specific for C4d and CD42b, respectively. Levels of C4d and CD42b were quantified by flow cytometry using a FACSCalibur cytometer (Becton Dickinson, Franklin Lakes, N.J.). The platelets were identified by forward and side scatter and CD42b fluorescence, and the mean fluorescence channel (MFC) was determined for C4d as well as for CD42b.

TABLE I
Healthy Controls (n = 25)
Mean MFC = −0.39
Range (−1.17) to (+0.87)

| Platelet C4d | MFC |
|---|---|
| 2003 | −0.28 |
| 2005 | −0.23 |
| 2006 | −0.51 |
| 2007 | −0.05 |
| 2008 | 0.20 |
| 2009 | 0.15 |
| 2010 | −0.39 |
| 2011 | −0.71 |
| 2013 | −0.96 |
| 2017 | 0.87 |
| 2020 | −0.29 |
| 2021 | −0.56 |
| 2022 | 0.38 |
| 2025 | −0.73 |
| 2026 | −0.24 |
| 2027 | −0.34 |
| 2028 | −0.74 |
| 2029 | −0.05 |
| 2030 | −0.51 |
| 2031 | −1.03 |
| 2032 | −0.42 |
| 2034 | −0.71 |
| 2035 | −0.86 |
| 2036 | −0.48 |
| 2037 | −1.17 |

More particularly, blood was drawn into 4 cc Vacutainer tubes containing 7.2 mg EDTA as an anticoagulant (Becton Dickinson), and processed within two hours. Whole blood was diluted 1/10 in phosphate buffered saline (PBS). Ten μl aliquots of the diluted blood were immufluorescently labeled for flow cytometry with 0.25 μg of PE-labeled anti-CD42b monoclonal antibody (Becton Dickinson Immunocytometry Systems, San Jose, Calif.) to identify platelets, and 0.25 μg of anti-C4d monoclonal antibodies (Quidel Corp., San Diego, Calif.) conjugated to Alexa Fluor 488 (Molecular Probes, Eugene, Oreg.) or the isotype control MOPC21 (obtained from American Type Culture Collection, Manassas, Va.). Samples were incubated 10 min at room temperature, then diluted with 0.5 ml cold PBS and analyzed on a FACS Calibur flow cytometer (Becton Dickinson Immunocytometry Systems, San Jose, Calif.). Platelets were electronically gated by forward scatter properties and expression of CD42b, a platelet-specific marker. Nonspecific binding of immunoglobulins to platelets was determined by performing identical assays in parallel using the isotype control antibody MOPC21. Specific binding of anti-C4d and anti-CD42b were determined by subtracting the MFC obtained with MOPC21 from the MFC obtained with anti-C4d and anti-CD42b, respectively.

### Example 2

### Platelet C4d as a Biomarker of Stroke Severity

This study was undertaken to determine the prevalence of platelet C4d in patients with ischemic stroke; and to determine the association between platelet C4d and stroke severity.

### 1. Materials and Methods

Eighty patients were recruited for this study. The diagnosis of ischemic stroke was determined by a neurologist based on neurological exam and evidence of acute infarct on MRI or CT. The National Institutes of Health Stroke Scale (NIH-SS) was assessed for each patient at the time of admission. Stroke types were classified into etiologic and localization subtypes. The etiologic subtypes included cardioembolic, large artery atherosclerosis, small vessel occlusion, stroke of other determined etiology, and stroke of undetermined etiology. Localization subtypes included large anterior circulation infarcts with both cortical and subcortical involvement (total anterior circulation infarcts), more restricted and predominantly cortical infarcts (partial anterior circulation infarcts), vertebrobasilar arterial territory (posterior circulation infarcts), and infarcts in the territory of the deep perforating arteries (lacunar infarcts). Demographic and clinical data including vascular risk factors, prior medical history, medication use, and neuroimaging were recorded for each patient.

Blood sample collections were initiated within 72 hours after hospital admission and when conditions permitted. Analyses included anticardiolipin (aCL) IgG and IgM antibodies, platelet C4d, and plasma C3 and C4 levels. The isotype specific measurements of aCL IgM and IgG antibodies were determined using the EL-aCL™ ELISA kit (TheraTest Labs, Inc., Illinois). Plasma C3 and C4 levels were measured by nephelometry (Beckman Coulter, Brea, Calif.). Platelet C4d was detected as previously described (Navratil, J. S. et al. Arthritis Rheum. 2006; 5(2):670-674). Briefly, whole blood was incubated with mouse monoclonal anti-human C4d antibody (Quidel Corporation, San Diego, Calif.) or a mouse IgGlk isotype control (Becton Dickinson, San Jose, Calif.) that were labeled with Alexa Fluor® 488 using a mouse IgG1 Zenon labeling kit (Molecular Probes, Eugene, Oreg.). The blood was then diluted and analyzed immediately by flow cytometry. Platelets were identified with a phycoerythrin (PE)-conjugated anti-CD42b antibody (Pharmingen, San Diego, Calif.). Based on a previous study, a cutoff value of 2.15 was determined for this assay. (Navratil, J. S. et al. Arthritis Rheum. 2006; 5(2):670-674). This cutoff took into account slight variations in fluorescence labeling between the MOPC21 isotype control and the anti-C4d antibodies, as well as the detection limitations of the flow cytometer. Platelet C4d-specific fluorescence intensity values of greater than or equal to 2.15 were considered to be positive for complement deposition.

All patients underwent either magnetic resonance imaging (MRI) or computed tomography (CT) of the brain within 48 hours of hospital admission. Two radiologists measured infarct volume by consensus: a third-year radiology resident (SF), and a board-certified neuroradiologist (BFB). Infarct volume was measured on fluid attenuation inversion recovery (FLAIR) sequences, using diffusion weighted sequences as a guide when acuity of infarct was indeterminate on FLAIR images alone. On each axial image, areas of infarction were outlined with a freehand region of interest, and a three dimensional volume was computed by summing the areas of infarct on each slice and multiplying by the slice interval (GE Advantage Workstation, Version 4.0). Following an overall measurement of infarct volume, the pre- and post-central sulci, thalamus, and basal ganglia were individually evaluated to determine whether 50% or more of each of these specific territories had infarcted. Finally, the brainstem was analyzed and any areas of infarction within the brainstem were noted.

Data were presented with mean (standard deviation) or median (interquartile range/IQR: 25^{th}-75^{th} percentile) based on the distribution of the continuous variables. Differences in categorical variables were analyzed using Fisher's Exact or Chi-square test. Spearman rank correlation was used to determine the correlation between two variables. The two stroke outcome variables of interest were stroke severity (as measured by NIH-SS) and infarct volume. NIH-SS scores and stroke volumes were transformed to normality by square root and log, respectively. Variables which had univariate association with stroke outcome variable at p≦0.15 entered the stepwise forward selection. Multivariable linear regression was utilized to assess for independent association of platelet C4d with each stroke outcome variable. All tests used a two-tailed significance level of 0.05. Analyses were performed using the STATA/SE version 9.0 for Windows (Stata Corporation, College Station, Tex.).

### 2. Results

The demographics and characteristics of the 80 acute ischemic stroke patients are shown in Table 2.

TABLE 2
Demographic and Clinical Characteristics of Patients*

| | | | Platelet C4d | | Platelet C4d | | |
|---|---|---|---|---|---|---|---|
| | All Stroke patients | | Positive | | Negative | | |
| | (n = 80) | | (n = 8) | | (n = 72) | | p-value^{†} |
| Demographic and risk | | | | | | | |
| factors | | | | | | | |
| Age | 57.9 | (14.2) | 61.7 | (12.2) | 57.5 | (14.4) | 0.43 |
| Sex (% male) | 46 | (57.5) | 6 | (75) | 40 | (55.6) | 0.46 |
| Race (% Caucasian) | 73 | (91.3) | 8 | (100) | 65 | (90.3) | 1.00 |
| Hypertension (%) | 58 | (72.5) | 7 | (87.5) | 51 | (70.8) | 0.43 |
| Diabetes (%) | 25 | (31.3) | 2 | (25) | 23 | (31.9) | 1.00 |
| Coronary heart disease (%) | 23 | (28.8) | 1 | (12.5) | 22 | (30.6) | 0.43 |
| Previous stroke (%) | 23 | (28.8) | 3 | (50) | 20 | (30.8) | 0.38 |
| Prior stroke by MRI (%) | 12/64 | (18.8) | 2/6 | (33.3) | 10/58 | (17.2) | 0.31 |
| Dyslipidemia (%) | 44 | (55) | 5 | (71.4) | 39 | (59.1) | 0.70 |
| Smoking ever (%) | 48 | (60) | 6 | (75) | 42 | (58.3) | 0.47 |
| Laboratory | | | | | | | |
| Blood collection (hr)^{‡} | 52.5 | (IQR 22.9-96) | 31 | (IQR 18.6-56.4) | 56.5 | (IQR 24-96) | 0.15 |
| WBC, mm³ × 10⁻³ | 9.8 | (IQR 7-11.1) | 10.3 | (IQR 10-11.9) | 8.9 | (IQR 6.4-11.1) | 0.09 |
| Platelet count, mm³ × 10⁻³ | 246.7 | (82.4) | 231 | (91) | 248 | (82) | 0.58 |
| Plasma C3 | 113.7 | (25.2) | 100.9 | (20.8) | 115.2 | (25.3) | 0.12 |
| Plasma C4 | 26.4 | (7.7) | 23.8 | (6.1) | 26.7 | (7.8) | 0.45 |
| Anticardiolipin | 18/79 | (22.5) | 1 | (12.5) | 17/71 | (23.9) | 0.67 |
| antibodies/aCL (%) | | | | | | | |
| aCL IgG | 2/79 | (2.5) | 0 | | 2/71 | (2.8) | 1.0 |
| aCL IgM | 18/79 | (22.5) | 1 | (12.5) | 17/71 | (23.9) | 0.67 |
| Stroke severity measures | | | | | | | |
| NIH-SS score | 6 | (IQR 2-13) | 17.5 | (IQR 9-21) | 5 | (IQR 2-11.5) | 0.003 |
| Infarct volume, cc | 3.4 | (IQR 1.1-16.6) | 17.4 (IQR 3.2-238.4) | | 2.9 (IQR 1.1-13.7) | | 0.06 |
| | | | n = 7 | | n = 59 | | |
| Ischemic Stroke | | | | | | | |
| Subtypes by TOAST | | | | | | | |
| criteria | | | | | | | |
| Cardioembolic | 12 | (15) | 2 | (25) | 10 | (13.9) | 0.34 |
| Large artery | 20 | (25) | 3 | (37.5) | 17 | (23.6) | 0.41 |
| atherosclerosis | | | | | | | |
| Small vessel occlusion | 18 | (22.5) | 2 | (25) | 16 | (22.2) | 1.0 |
| Stroke of other | 8 | (10) | 0 | | 9 | (12.5) | 0.59 |
| determined etiology | | | | | | | |
| Stroke of undetermined | 21 | (26.6) | 1 | (12.5) | 20 | (27.8) | 0.67 |
| etiology | | | | | | | |
| Stroke location | | | | | | | |
| Total anterior | 9 | (11.3) | 4 | (50) | 4 | (6.9) | 0.004 |
| Partial anterior | 34 | (42.5) | 1 | (12.5) | 33 | (45.8) | 0.13 |
| Posterior | 15 | (18.8) | 1 | (12.5) | 14 | (19.4) | 1.0 |
| Lacunar | 18 | (22.8) | 2 | (25) | 16 | (22.2) | 1.0 |
| Acute stroke therapy | | | | | | | |
| Thrombolytics | 23 | (28.8) | 5 | (62.5) | 18 | (25) | 0.02 |
| tPA (%) | 21 | (26.3) | 4 | (50) | 17 | (23.6) | 0.10 |
| Urokinase (%) | 2 | (2.5) | 1 | (12.5) | 1 | (1.4) | 0.19 |
| Platelet glycoprotein | | | | | | | |
| IIb/IIIa inhibitor | | | | | | | |
| Abciximab (%) | 2 | (2.5) | 0 | | 2 | (2.8) | 1.0 |
| Eptifibatide (%) | 4 | (5) | 2 | (25) | 2 | (2.8) | 0.02 |
| Dipyridamole (%) | 20 | (25) | 4 | (50) | 16 | (22.2) | 0.05 |
| Heparin (%) | 60 | (75) | 5 | (62.5) | 55 | (76.4) | 0.30 |

*Continuous variables are presented as mean (standard deviation) or median (Interquartile range/IQR: 25^{th}-75^{th} percentile) depending on the data distribution. Stroke volume measurement was available for 66 patients. t-PA = tissue plasminogen activator
^{†}Comparison between patients with moderate to severe stroke and those with mild stroke, statistical significance at p < 0.05
^{‡}Time of blood draw from symptom onset

The overall mean age of the ischemic stroke patients was 57.9 years (range: 24.6-86.8 years) with 30% aged less than 50 years and more than half (58%) of these patients were male. Median NIH-SS score was 6 (IQR: 2-13) and median infarct volume was 3.4 cc (IQR: 1.1-16.6). There was no significant difference in the demographics, cardiovascular risk factors, and medications (i.e. antiplatelet and anticoagulation therapy) at home and during hospitalization between patients with positive platelet C4d and those with negative platelet C4d. However, patients with positive platelet C4d were more likely to have received thrombolytics than platelet C4d-negative patients (p=0.02).

Peripheral venous blood samples were collected at a median time of 52.5 hours (interquartile range: 22.9-96) after the onset of stroke symptoms. Patients with positive platelet C4d appeared to have a shorter interval between onset of stroke symptoms and blood collection time although this difference did not reach statistical significance (p=0.15).

In the eighty stroke patients, at initial blood collection, the median platelet C4d level was 0.45 (interquartile range from 0.14 to 0.79). Six out of eighty patients (7.5%) had a positive platelet C4d level at initial blood collection (cut off level=2.15) with two additional patients having positive platelet C4d levels during subsequent blood collections, totaling eight out of eighty patients (10%) with positive platelet C4d levels during their hospitalization (shown in Table 3).

TABLE 3
Stroke patients with Positive platelet C4d levels

| Identification Number | Visit Number | pC4d level |
|---|---|---|
| 23001 | 1 | 2.52 |
| 23010 | 1 | 16.53 |
| 23014 | 1 | 6 |
| 23020 | 1 | 7.76 |
| 23025 | 1 | 2.7 |
| 23026 | 1 | 2.25 |
| 23064 | 1 | 1.71 |
| 23064 | 2 | 1.36 |
| 23064 | 3 | 2.45 |
| 23064 | 4 | 1.44 |
| 23064 | 5 | 1.12 |
| 23064 | 6 | 1.53 |
| 23072 | 1 | 0.92 |
| 23072 | 2 | 1.71 |
| 23072 | 3 | 2.18 |
| 23072 | 4 | 0.53 |
| 23072 | 5 | 0.21 |

Eight patients who had positive platelet C4d had significantly more severe stroke (NIH-SS median: 17.5 vs. 5, p=0.003) and greater infarct volume (median: 17.4 cc vs. 2.9 cc, p=0.06) than those with negative platelet C4d. Their mean age was 61.7 years (range: 46.3 to 83.9 years) and 75% were male. The majority (6/8) of these patients with positive platelet C4d had only single blood collection; the two patients with serial blood collection had initially negative platelet C4d and then became positive platelet C4d in subsequent blood collections. The remaining 16 patients with sequential blood collections exhibited consistently negative platelet C4d.

Nearly a quarter of stroke patients (22.5%) had positive aCL antibodies. Only one patient with aCL-positivity was also positive for platelet C4d; his platelet C4d value (16.5) was the highest of all patients studied. Three patients had autoimmune disease (systemic lupus erythematosus/SLE, rheumatoid arthritis/RA, and Takayasu's arteritis). Both the SLE and RA patients had moderate-to-severe stroke, while the patient with Takayasu's arteritis had mild stroke. Only the SLE patient was positive for platelet C4d (2.52). The RA patient was positive for aCL antibodies. Although the patient with Takayasu's arteritis did not test positive for aCL antibodies in this study, she had prior history of positive aCL antibodies. There were two other patients who also had a history of antiphospholipid antibodies. One patient had both positive aCL and lupus anticoagulant and continued to test positive for aCL antibodies in this study, whereas the other patient with a history of positive lupus anticoagulant tested negative for aCL antibodies. One of the 8 patients with positive platelet C4d also had positive aCL antibodies; this patient had the highest platelet C4d level of 16.5. This was the only patient positive for both platelet C4d and aCL antibodies.

Using Spearman's rank correlations, platelet C4d correlated with stroke severity by NIH-SS (rₛ=0.34, p=0.002) and infarct volume (rₛ=0.24, p=0.06), as shown in Table 4.

TABLE 4
Clinical Correlates of Stroke Severity using Spearman Rank
Correlation

| | |
|---|---|
| NIH-SS | Infarct Volume |
| (n = 80) | (n = 66) |

| | rₛ | p-value | rₛ | p-value |
|---|---|---|---|---|
| Age | 0.28 | 0.01 | 0.07 | 0.57 |
| Old stroke by MRI | −0.19 | 0.13 | −0.26 | 0.04 |
| WBC | 0.04 | 0.74 | 0.08 | 0.51 |
| Hemoglobin | −0.28 | 0.01 | −0.09 | 0.46 |
| Platelet count | −0.26 | 0.03 | 0.09 | 0.49 |
| Platelet C4d positive | 0.34 | 0.002 | 0.24 | 0.06 |
| Plasma C3 | −0.33 | 0.003 | −0.18 | 0.14 |
| Plasma C4 | −0.22 | 0.05 | −0.22 | 0.08 |
| aCL antibody positive | 0.18 | 0.12 | 0.26 | 0.04 |
| Ischemic stroke | | | | |
| subtypes | | | | |
| Cardioembolic | 0.27 | 0.01 | 0.15 | 0.21 |
| Large-vessel | 0.17 | 0.13 | 0.36 | 0.003 |
| Small-vessel | −0.18 | 0.10 | −0.45 | <0.001 |
| Location of stroke | | | | |
| Total anterior | 0.44 | <0.001 | 0.35 | 0.004 |
| Lacunar | −0.14 | 0.21 | −0.43 | <0.001 |

NIH-SS was moderately correlated with infarct volume (rₛ=0.56, p<0.0001). Platelet C4d did not correlate with either plasma C3 (rₛ=−0.13, p=0.27) or C4 (rₛ=−0.05, p=0.69). Age, hemoglobin, platelet count, plasma C3, cardioembolic subtype of ischemic stroke and location of stroke (total anterior circulation) were correlated with stroke severity by NIH-SS. Meanwhile, aCL antibodies, large-vessel and small-vessel ischemic stroke subtypes, and stroke locations (total anterior and lacunar infarct) were significantly correlated with infarct volume.

As shown in Table 5, using multivariable linear regression after forward stepwise selection, platelet C4d positivity (beta coefficient=1.05, p=0.03) was independently associated with stroke severity by NIH-SS after adjusting for age, presence of aCL antibodies and location of stroke (total anterior circulation). Similarly, platelet C4d positivity (beta coefficient=2.61, p=0.005) also was associated with infarct volume after adjusting for age, presence of aCL antibodies and evidence of prior stroke by MRI.

TABLE 5
Age-adjusted association of platelet C4d with stroke severity by
NIH stroke scale and infarct volume using multivariable linear
regression*

| | NIH Stroke Scale | | | Infarct Volume | | |
|---|---|---|---|---|---|---|
| | (R² = 0.30) | | | (R² = 0.26) | | |
| Independent | β | | | β | | |
| variables | coefficient | SE | p | coefficient | SE | p |
| P-C4d positivity | 1.05 | 0.48 | 0.03 | 2.61 | 0.90 | 0.005 |
| aCL positivity | 0.66 | 0.32 | 0.045 | 1.36 | 0.64 | 0.04 |
| Total anterior | 1.38 | 0.47 | 0.004 | | | |
| circulation | | | | | | |
| involvement | | | | | | |
| of stroke | | | | | | |
| Old stroke by MRI | | | | −1.89 | 0.67 | 0.006 |

*Known risk factors for stroke such as hypertension, dyslipidemia, smoking history, and diabetes did not contribute to the final multivariable regression models

Platelet C4d positivity (beta coefficient=2.06, p=0.02) continued to be significantly associated with infarct volume after additional adjustment for the large-vessel subtype of ischemic stroke and lacunar infarcts. However, the presence of aCL antibodies (beta coefficient=1.12, p=0.07) lost its association with infarct volume after this additional covariate adjustment. There was no interaction between platelet C4d positivity and presence of aCL antibodies or prior stroke by MRI. After excluding the one SLE patient with positive platelet C4d, the association of positive platelet C4d with stroke severity by NIH-SS (beta coefficient=1.03, p=0.04) and infarct volume (beta coefficient=2.22, p=0.03) remained statistically significant. The results also remained the same after excluding all three patients with rheumatologic diseases.

### 3. Discussion

This study identified two significant findings. First, C4d deposits on the surface of platelets in some patients with acute ischemic stroke. Second, positive platelet C4d is associated with stroke severity by NIH-SS and infarct volume in these patients. Even though the prevalence of positive platelet C4d was only 10% in our cohort of acute ischemic stroke patients, it identified a subset of patients with greater cerebrovascular injury after acute ischemic stroke. These findings corroborate previous work that demonstrated the involvement of complement in acute ischemic stroke (Di Napoli M., Stroke, 2001; 32(6):1443-1448; Atkinson, C. et al., J. Immunol., 2006; 177(10):7266-7274; Mocco J. et al., Circ. Res., 2006; 99(2):209-217; Pedersen, E. D. et al., Clin. Exp. Immunol., 2004; 137(1):117-122; Mocco, J. et al., Neurosurgery, 2006; 59(1):28-33; Figueroa E. et al., Neurosci. Lett., 2005; 380(1-2):48-53; 1 mm, M. D. et al., Neurosci. Lett., 2002; 325(3):175-178) and also suggest a novel interaction between complement activation and platelets in cerebral ischemic injury.

Antiphospholipid antibodies are known risk factors for vascular occlusive disorders and recurrent fetal loss. In a previous study, it was demonstrated that platelet C4d was independently associated with the antiphospholipid antibodies [aCL and lupus anticoagulant (LAC)] in SLE patients (Navratil, J. S. et al., Arthritis Rheum., 2006; 54(2):670-674). Several studies also have shown the presence of antiphospholipid antibodies (aCL and LAC) as a stroke risk factor in young adults (maximum age cutoffs ranged from 40 to 51 years), primarily in patients without SLE (Brey, R. L., et al., Editorial Comment, Stroke. 2002; 33(10):2396-2401; Brey, R., et al., Neurology, 1990; 40(8):1190-1196; Nencini. P. et al., Stroke, 1992; 23(2):189-193; Toschi, V., et al., Stroke, 1998; 29(9):1759-1764; Singh, K. et al., J. Assoc. Physicians India, 2001; 49:527-529). This study demonstrated the presence of aCL antibodies in 22.5% of the acute ischemic stroke patients, similar to that previously reported in the literature. In addition, two of the three patients with known positivity for aCL and/or LAC tested negative for aCL in this study. Although LAC was not measured, and most of the aCL antibodies detected were of IgM isotype, it was found that aCL was independently associated with higher infarct volume. More importantly, this study demonstrated an independent positive association of platelet C4d with stroke severity by NIH-SS and infarct volume even after adjusting for covariates and/or potential confounders which included aCL, stroke subtype and location. Taken together, in a subset of patients, platelet C4d is a potential biomarker of ischemic stroke injury and may have a different role from that of aCL.

In conclusion, there is an important unmet need to find biomarkers to identify patients with acute ischemic stroke at risk for severe events and associated morbidity. This study demonstrated that C4d is another participant of complement activation in ischemia-reperfusion injury after acute stroke and that platelet C4d appears to be a biomarker in identifying a subset of acute ischemic stroke patients with greater stroke severity. Together, these observations suggest that platelet C4d may not only be a biomarker but also a pathogenic marker that links complement activation, cerebrovascular injury and thrombosis.

While the invention has been particularly shown and described with reference to preferred embodiments thereof, it will be understood by those skilled in the art that various alterations in form and detail may be made therein without departing from the spirit and scope of the invention, as defined by the appended claims.

What is claimed is:

1. A method for assessing risk of cerebrovascular thrombosis in an individual comprising the steps of:
(a) determining the platelet surface level of a complement pathway component C4d in the individual, and
(b) comparing the level of C4d of the individual with a standard control, wherein an increase in the level of C4d from the standard control indicates an increased risk of cerebrovascular thrombosis in the individual.

2. The method of claim 1, wherein the level of C4d is determined using an antibody that specifically binds C4d.

3. The method of claim 1, wherein the C4d antibody is labeled.

4. The method of claim 3, wherein the C4d antibody is labeled with a fluorescent moiety.

5. The method of claim 3, wherein the C4d antibody is detected by flow cytometry.

6. The method of claim 1, wherein the individual has no clinical symptoms of cerebrovascular thrombosis.

7. The method of claim 1, wherein the individual has previously suffered from cerebrovascular thrombosis.

8. A method for diagnosing or monitoring cerebrovascular thrombosis in an individual comprising the steps of:
(a) determining the platelet surface level of a complement pathway component 4 C4d in the individual, and
(b) comparing the level of C4d of the individual with a standard control, wherein an increase in the level of C4d from the standard control indicates the presence of cerebrovascular thrombosis or a worsening of cerebrovascular thrombosis in the individual.

9. The method of claim 8, wherein the level of C4d is determined using an antibody that specifically binds C4d.

10. The method of claim 9, wherein the C4d antibody is labeled.

11. The method of claim 10, wherein the C4d antibody is labeled with a fluorescent moiety.

12. The method of claim 10, wherein the C4d antibody is detected by flow cytometry.

13. The method of claim 8, wherein the individual has one or more clinical symptoms of cerebrovascular thrombosis.

14. A method of assessing the severity of cerebrovascular thrombosis in an individual, comprising the steps of:
(a) determining the platelet surface level of a complement pathway component C4d in the individual; and
(b) comparing the platelet surface level of the complement pathway component C4d of the individual with a standard control, wherein the magnitude of increase in the surface level of the complement pathway component C4d of the individual from the standard control correlates with the severity of cerebrovascular thrombosis in the individual.

15. The method of claim 14, wherein the level of C4d is determined using an antibody that specifically binds C4d.

16. The method of claim 15, wherein the C4d antibody is labeled.

17. The method of claim 16, wherein the C4d antibody is labeled with a fluorescent moiety.

18. The method of claim 16, wherein the C4d antibody is detected by flow cytometry.

19. The method of claim 14, wherein the individual has one or more clinical symptoms of cerebrovascular thrombosis.

20. A computer readable medium for predicting, diagnosing, or monitoring cerebrovascular thrombosis in an individual, the computer readable medium comprising:
(a) code for receiving data corresponding to a level of the complement pathway component C4d on the surface of platelets from an individual;
(b) code for retrieving a standard control; and
(c) code for comparing the data in (a) with the standard control in (b).
